(19) United States
(12) Patent Application Publication
(10) Pub. No.: US 2005/0053669 A1
(43) Pub. Date: Mar. 10, 2005

(54) Administration form for the oral application of poorly soluble acidic and amphorteric drugs

(75) Inventors: Thomas Friedl, Ochsenhausen (DE); Rolf-Stefan Brickl, Warthausen (DE); Ulrich Brauns, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 10/925,313

(22) Filed: Aug. 24, 2004

Related U.S. Application Data
(60) Provisional application No. 60/514,997, filed on Oct. 28, 2003.

(30) Foreign Application Priority Data
Sep. 5, 2003 (DE) DE 103 41 414

(51) Int. Cl. A61K 9/16; A61K 9/50

(52) U.S. Cl. 424/490

(57) ABSTRACT

The invention relates to a new formulation for oral administration of active substances with pH-dependent solubility characteristics and the pharmacologically acceptable salts thereof, which improves the bioavailability of the active substance.

### RELATED APPLICATIONS

Benefit of U.S. Provisional Application Ser. No. 60/514,997, filed on Oct. 28, 2003 is hereby claimed, and which application is incorporated herein in its entirety.

### FIELD OF THE INVENTION

The invention relates to a formulation for the oral administration of acidic and amphoteric active substances with pH-dependent solubility characteristics, and the salts thereof.

The term “active substance” for the purposes of this invention refers to any pharmacologically effective compound which (as such or in the form of the pharmaceutically acceptable salts thereof) is a weak acid or behaves amphoterically and in the range from pH 1 to pH 11 exhibits pH-dependent solubility characteristics (with greater solubility under basic conditions and less solubility under neutral and/or acidic conditions). Acidic active substances are preferred. In these active substances, in fact, the bioavailability may be dependent on the pH in the gastrointestinal tract when administered orally. Preferably, active substances in the sense of this invention have a relatively high saturation solubility in aqueous solutions at higher pH levels, whereas at pH values of about 5 they are virtually insoluble according to the definition in the European Pharmacopoeia (saturation solubility less than 100 μg/ml).

The oral formulation according to the invention may contain as active substance for example telmisartan, meloxicam, DT-TX 30 SE, BIBV 308 SE (terbogrel), AGEE 623 (repaglinide), gliquidone or glibenclamide or a physiologically acceptable salt thereof. The physiologically acceptable salts include for example the sodium, potassium, calcium and ammonium salt as well as salts with diethanolamine, meglumine, lysine, arginine, ethanolamine, piperazine or triethanolamine.

The solubility of a compound may be determined by dispersing an excess of the compound at ambient temperature in the medium in question and shaking it vigorously for a defined length of time (approx. 1 to 24 h) until equilibrium is achieved. After filtration the pH is determined in the clear filtrate and the concentration of the dissolved substance is determined by spectral photometry or some other suitable analytical process.

The pH-dependent solubility characteristics of the active substance may mean that, depending on the dose, when administered orally in solid preparations of conventional composition, the active substance is only totally dissolved when it reaches lower parts of the patient's intestines. This then leads to significantly delayed and in some cases incomplete absorption, i.e. there is no therapeutic certainty.

The effect of the dose of the active substance on its bioavailability can be quantitatively described by means of the concept of the (dimensionless) dose number (Do). The dose number is defined as:
Do=(Mo/Vo)/Cs,
where
Mo=dose (mg),
Vo=liquid volume present (ml) and
Cs=saturation solubility (mg/ml).

According to an assumption which is conventional nowadays the liquid volume in the stomach after taking a preparation is about 250 ml. (Löbenberg, R., Amidon, G. L.: Modern bioavailability, bioequivalence and biopharmaceutics classification system. New scientific approaches to international regulatory standards (Eur. J. Pharm. Biopharm. 50 (2000) 3-12).

At dosages which give a dose number of less than 1, no solubility problems occur. Only if the critical dose number of 1 is exceeded may there be significant reductions in solubility and hence a decreased bioavailability. As a rule the actual problem area only begins at doses which give a dose number significantly above 1, as at least some of the dissolved substance can be constantly eliminated from the equilibrium by the absorption process.

The active substances contained in the oral formulation according to the invention have a dose number significantly higher than 1, based on the solubility in the range from pH 1 to pH 7 for acidic active substances or in the range from pH 3 to 7 for amphoteric active substances, i.e. for the oral formulation according to the invention both the degree of pH-dependence of the solubility of the active substance and the size of the dose of active substance are of interest. The formulation according to the invention is particularly suitable for active substances which have poor solubility below pH 6 according to the dose number defined above.

Generally increasing the dose in order to compensate for the reduced bioavailability in patients is frequently undesirable because of the waste of active substance and the greater burden on the patient and the associated risk of side effects, for example, or even totally impossible, on the grounds of drug safety. In any case, with active substances which have poor solubility increasing the dose does not necessarily lead to the expected plasma levels: in the case of DT-TX 30, in a phase I study only about 50% higher plasma levels were obtained by increasing the dose from 100 to 600 mg, and levels equal to those obtained with 600 mg were achieved with 200 mg.

The aim of the invention is to provide a pharmaceutical composition for oral administration of active substances with pH-dependent solubility characteristics which guarantees largely pH-independent bioavailability of the active substance.

Surprisingly, it has now been found that the use of pharmaceutically acceptable inorganic or organic bases with a water solubility of more than 1 g/250 ml at 20° C., preferably more than 1 g/160 ml at 25° C., in solid oral formulations can ensure sufficient bioavailability of active substances with pH-dependent solubility characteristics. Organic bases and mixtures of inorganic and organic bases are preferred.

Pharmaceutically suitable bases for the purposes of this invention are for example sodium hydroxide (NaOH), potassium hydroxide (KOH), calcium hydroxide [Ca(OH)₂], sodium carbonate (Na₂CO₃), ammonia, diethanolamine, meglumine, lysine, arginine, ethanolamine, piperazine, trometamol and triethanolamine. NaOH, KOH, Ca(OH)₂, ammonia, diethanolamine, meglumine, lysine and arginine are particularly suitable for the purposes of this invention. Most particularly preferred are meglumine, lysine, arginine and trometamol. The advantage of the inorganic bases is the low molecular weight, which makes it possible to produce formulations with very high contents of active substance, while the ratio of base to active substance must be selected so that no irritation occurs in the stomach or intestines as a result of higher pH levels. If desired, inorganic and organic bases may also be combined so as to achieve physiologically acceptable pH values of not more than 12.

Numerous active substances display a more or less marked tendency to hydrolytic decomposition in the presence of bases and traces of water. In individual cases there may even be a direct chemical reaction between the active substance and bases, e.g. ester formation. When developing a product which remains stable when stored it is therefore advantageous to separate the base spatially from the active substance in the formulation. Only after the administration of the formulation does the base dissolve and produce a basic microclimate in which the active substance can dissolve.

A further aim of the invention is to prevent the undesirable interactions between base and active substance in spite of the use of a base to improve the solubility.

Multiparticulate formulations in which the individual particles have the structure shown in FIG. 2 are particularly suitable for the preferred spatial separation of active substance and base.

FIG. 2 shows the diagrammatic structure of the pharmaceutical composition by means of a section through a pellet which is suitable for producing the pharmaceutical composition according to the invention. The roughly spherical core portion of this pellet contains or consists of one or more pharmaceutically acceptable inorganic or organic bases. This is optionally followed by a layer which separates the base-core from the layer containing the active substance, the so-called insulating layer. The insulating layer in turn, or the core material in the absence of an insulating layer, is surrounded by the active substance layer, which is also spherical, which may itself be surrounded by a coating to increase the abrasion resistance and shelf life of the pellets.

One advantage of a formulation in the form of pellets is the possibility of individual dosing, which is important when medicating children, for example.

The core material used is a pharmaceutically acceptable base with a water solubility of >1 g/250 ml at 20° C., such as e.g. NaOH, KOH, Ca(OH)₂, Na₂CO₃, ammonia, diethanolamine, meglumine, lysine, arginine, ethanolamine, piperazine, trometamol or triethanolamine, or mixtures of such bases, to which a small amount of 1 to 10% by weight, preferably 3 to 6% by weight of a suitable binder is optionally added. The use of a binder may be necessary, for example, if the core material is produced by a pan build-up process. If the method used is extrusion or spheronisation, other adjuvants such as microcrystalline cellulose will be needed instead of binders, optionally together with binders for increasing the mechanical stability. It is also possible to use pure (100%) base as the starting material if it can be obtained in a sufficiently narrow range of particle sizes. The pharmaceutically acceptable bases used are preferably NaOH, KOH, Ca(OH)₂, ammonia, diethanolamine, meglumine, lysine, arginine or trometamol; particularly preferred are meglumine, lysine, arginine and trometamol or mixtures thereof; meglumine is particularly preferred. As binder, it is possible to use gum arabic or a partially or totally synthetic polymer selected from among the hydroxypropylcelluloses, hydroxypropylmethylcelluloses, methylcelluloses, hydroxyethylcelluloses, carboxymethylcelluloses, polyvinylpyrrolidones, the copolymers of N-vinylpyrrolidone and vinyl acetate, or combinations of these polymers; gum arabic is preferred. The spherical core material preferably has an average diameter of 0.4-1.5 mm. The content of the pharmaceutically acceptable inorganic or organic base is usually between 30 and 100% in the core material. This base-containing spherical core material is also referred to as starter pellets or starter, for short (e.g. meglumine starter, lysine starter).

To increase the durability of the finished product, in the case of active substances which are unstable in the presence of bases, it is advantageous to coat the core material before the application of the active substance with an insulating layer based on a water-soluble, pharmaceutically acceptable polymer. Examples of such water-soluble polymers include for example gum arabic or a partially or totally synthetic polymer selected from among the hydroxypropylcelluloses, hydroxypropylmethylcelluloses, methylcelluloses, hydroxyethylcelluloses, carboxymethylcelluloses, polyvinylpyrrolidones, the copolymers of N-vinylpyrrolidone and vinyl acetate, or combinations of these polymers. Gum arabic or a hydroxypropylmethylcellulose is preferably used. If desired, the coating with the water-soluble, pharmaceutically acceptable polymer may be carried out with the addition of suitable plasticisers, separating agents and pigments, such as for example triethylcitrate, tributylcitrate, triacetin, polyethyleneglycols (plasticisers), talc, silicic acid (separating agents), titanium dioxide or iron oxide pigments (pigments).

The active substance layer contains the active substance as well as binders and optionally separating agents. Suitable binders include for example hydroxypropylcellulose, hydroxypropylmethylcellulose, methylcellulose, hydroxyethylcellulose, carboxymethylcellulose, polyvinylpyrrolidone, copolymers of N-vinylpyrrolidone and vinyl acetate or combinations of these polymers. Preferably, hydroxypropylcellulose or copolymers of N-vinylpyrrolidone and vinyl acetate are used. The addition of separating agents such as e.g. talc, magnesium stearate or silicic acid serves to prevent the particles from aggregating during the process. The active substance content is not more than 60%, preferably not more than 50% of the pharmaceutical composition.

The optional outermost layer, which serves to reduce any increased abrasion during packing into capsules and/or to increase the shelf life, consists of pharmaceutically conventional film-forming agents, plasticisers and optionally pigments. Suitable film-forming agents include for example hydroxypropylcellulose, hydroxypropylmethylcellulose, methylcellulose, polymers and copolymers of acrylic and methacrylic acid and the esters thereof, or combinations of these polymers. Suitable plasticisers include inter alia triethylcitrate, tributylcitrate, triacetin or polyethyleneglycols. The pigments used may be e.g. titanium dioxide or iron oxide pigments. Preferably, the outer coating consists of hydroxypropylmethylcellulose and/or methylcellulose, optionally with the addition of polyethyleneglycols as plasticisers.

The pellets may be prepared by the method described hereinafter:

The base-containing core material consists either of crystals of the particular base used or, more advantageously, of roughly spherical particles of the desired size containing a large amount of base, which can be produced by methods known and established in pharmaceutical technology. The core material may be produced, in particular, by pan methods, on pelleting plates or by extrusion/spheronisation. Then the core material thus obtained may be divided into fractions of the desired diameter by screening. Suitable core material has an average diameter of 0.4 to 1.5 mm, preferably 0.6 to 0.8 mm.

First, the insulating layer is applied to this base-containing core material. This can be done by conventional methods, e.g. by applying an aqueous dispersion of the water-soluble, pharmaceutically acceptable polymer, optionally with the addition of plasticisers, separating agents and/or pigments, in a fluidised bed, in coating pans or in conventional film coating apparatus. If necessary the product can then be screened again. Then the active substance is applied from a dispersion containing binder and optionally separating agent. The volatile dispersant is removed during and/or after the process by drying. Suitable binders in the dispersion may be for example hydroxypropylcellulose, hydroxypropylmethylcellulose, methylcellulose, hydroxyethylcellulose, carboxymethylcellulose, polyvinylpyrrolidone, copolymers of N-vinylpyrrolidone and vinyl acetate or combinations of these polymers. Preferably, hydroxypropylcellulose or copolymers of N-vinylpyrrolidone and vinyl acetate are used. Suitable separating agents include e.g. talc, magnesium stearate or silicic acid; preferably, talc is used. The dispersants may be for example water, ethanol, 2-propanol, acetone or mixtures of these solvents with one another. The application of active substance to the core material may be carried out by established methods known in pharmaceutical technology, e.g. in coating pans, conventional film coating apparatus or by the fluidised bed method. Then a further screening process may be carried out.

To reduce any increased abrasion during transfer into capsules or to increase the shelf life the system may finally be coated with a coating of a pharmaceutically conventional film forming agent, plasticiser and optionally pigment. This may be done by conventional methods as mentioned earlier in the description of the application of the insulating layer.

When core material with an average diameter of 0.4-1.5 mm is used, the process described above produces pellets containing active substance, which can then be packed into hard capsules, for example. To do this, a number of these units corresponding to the required dosage are packed into hard capsules in a standard capsule filling machine. Suitable hard capsules include, for example, hard gelatine capsules or hard capsules of hydroxypropylmethylcellulose (HPMC). The preferred active substance content of the pharmaceutical composition is not more than 60%, preferably not more than 50%.

Unless otherwise stated, percentages specified are always percent by weight. All the data on the active substance content relate to the active substance acid (not to a specific salt) unless otherwise stated.

The amount of active substance per capsule is preferably such that 1 to 2 capsules a day are sufficient to produce the desired activity.

For repaglinide, for example, daily doses of 0.2 mg to 5 mg, preferably capsules containing 0.5 mg, 1.0 mg or 2.0 mg are suitable.

For telmisartan, for example, daily doses of 10 mg to 150 mg, preferably capsules containing 20 mg, 40 mg or 80 mg are suitable.

For meloxicam, for example, daily doses of 4 mg to 30 mg, preferably capsules containing 7.5 mg or 15.0 mg are suitable.

For DT-TX 30, for example, daily doses of 50 mg to 300 mg, preferably capsules containing 100 mg or 200 mg are suitable.

For gliquidone, for example, daily doses of 10 mg to 50 mg, preferably capsules containing 30 mg are suitable.

For glibenclamide, for example, daily doses of 1.9 mg to 5.0 mg, preferably capsules containing 3.5 mg are suitable.

The preferred ratio of base to active substance is approx. 1:1 to approx. 20:1. The theoretical lower limit at which the system can still function is 1 equivalent of base per mol of active substance. The upper limit of approx. 20:1 (base to active substance) is determined by the size of the formulation at the desired doses (number of pellets per capsule).

In quality control, in vitro releases are measured by USP methods. The drug is released in a volume of 900 ml and the pH is selected so as to obtain “sink conditions”, i.e. the entire dose of active substance is soluble in these 900 ml. This in vitro method cannot be predictive of absorption in humans in most cases as a patient will generally take the drug with approx. 200 ml of liquid and in a non-acidic stomach the solubility is often only sufficient for a fraction of the dose. Non-acidic stomach occurs at a rate of about 25% of the population in older patients and is often also caused by co-medication with H2 blockers or proton pump inhibitors.

Therefore, within the scope of the invention, an empirical test method was developed which has a better correlation with the in vivo performance in humans. In this procedure, a drug preparation which contains the maximum dose used in humans is released in a volume of 200 ml (this corresponds to the dose in humans) in buffer at a pH with reduced solubility of the active substance in the physiologically acceptable range, i.e. between pH 1-7. As the absorbability can also be predicted with some accuracy using this method, even at non-acidic gastric pH levels, it is suitable for optimising drug preparations. In order to identify the most favourable formulation in each case from a number of possible recipes, the maximum release and/or the area under the curve (AUC) from time 0 to the end of the release may be used as relevant characteristics.

This is clear from the example of the comparison of the formulation examples c1-c2 (reference to neutral starter) and c3-c5 (Example according to the invention with meglumine starter) (FIG. 3 and Table 1). By “neutral starter”, neutral core” and neutral pellet” are meant in each case standard commercial pellets of sucrose or microcrystalline cellulose.

Table 1 a shows the in vitro releases and the characteristics of the area under the release curve (AUC) and the maximum release of Examples c3-c5 according to the invention (active substance: telmisartan) compared with the reference forms of Examples c1 and c2 with an even lower content of active substance in 0.005 mol citrate buffer pH 5.0

TABLE 1a
Comparison of the in vitro releases of pellets on
base starters (Examples c3-c5) and neutral starters
(Examples c1-c2)

| | content of | in vitro | | | | | | | |
|---|---|---|---|---|---|---|---|---|---|
| | active | release after . . . minutes | | | | | | | maximum |
| Example | substance | 0 | 4 | 8 | 12 | 16 | 20 | AUC | release |
| c1 | 2.6 | 0 | 0 | 0 | 0 | 0 | 0 | 0 | 0 |
| c2 | 16.8 | 0 | 0 | 0 | 0 | 0 | 0 | 0 | 0 |
| c3 | 8.9 | 0 | 44 | 50 | 62 | 72 | 69 | 1046 | 72 |
| c4 | 23.2 | 0 | 18 | 14 | 17 | 17 | 14 | 293 | 18 |
| c5 | 29.4 | 0 | 9 | 9 | 8 | 8 | 6 | 147 | 9 |

Other Examples are the comparisons of formulation examples c28 (reference to neutral starter) with c31, c32 (Example according to the invention with meglumine starter) and c33-c35 (Example according to the invention with arginine starter) as well as the comparisons of formulation examples c43 and c45 (reference to neutral starter) with c46 and c47 (Example according to the invention with meglumine starter) and c49 and c49a (Example according to the invention with arginine starter).

Table 1b shows the in vitro releases and the characteristics of area under the release curve (AUC) and maximum release of the Examples c31, c32, c33, c34 and c35 according to the invention (active substance: meloxicam) compared with the reference form of Example c28 in 0.01 mol phosphate buffer pH 5.0

TABLE 1b
Comparison of the in vitro releases of pellets on base
starters (Examples c31, c32, c33, c34, c35) and neutral
starters (Example c28)

| | content of | in vitro | | | | | | |
|---|---|---|---|---|---|---|---|---|
| | active | release after . . . minutes | | | | | | maximum |
| Example | substance | 0 | 5 | 10 | 15 | 20 | AUC | release |
| c28 | 16.5 | 0 | 17 | 6 | 3 | 0 | 126 | 17 |
| c31 | 16.8 | 0 | 71 | 83 | 87 | 88 | 1426 | 88 |
| c32 | 26.1 | 0 | 58 | 61 | 62 | 52 | 1034 | 62 |
| c33 | 17.1 | 0 | 55 | 57 | 52 | 53 | 951 | 57 |
| c34 | 28.6 | 0 | 34 | 37 | 41 | 36 | 652 | 41 |
| c35 | 33.3 | 0 | 34 | 36 | 35 | 29 | 596 | 36 |

Table 1c shows the in vitro releases and the characteristics of area under the release curve (AUC) and maximum release of Examples c46, c47, c49 and c49a according to the invention (active substance: DT-TX 30) compared with reference forms of Examples c43 and c45 in 0.025 mol phosphate buffer pH 6.0

TABLE 1c
Comparison of the in vitro releases of pellets on base
starters (Examples c46, c47, c49 and c49a and neutral
starters (Examples c43, c45)

| | content of | in vitro | | | | | | | |
|---|---|---|---|---|---|---|---|---|---|
| | active | release after . . . minutes | | | | | | | maximum |
| Example | substance | 0 | 4 | 8 | 12 | 16 | 20 | AUC | release |
| c43 | 15.8 | 0 | 7 | 6 | 5 | 0 | 0 | 74 | 7 |
| c45 | 31.2 | 0 | 7 | 8 | 0 | 0 | 0 | 58 | 8 |
| c46 | 16.3 | 0 | 19 | 51 | 55 | 56 | 58 | 845 | 58 |
| c47 | 25.7 | 0 | 8 | 35 | 38 | 39 | 39 | 558 | 39 |
| c49 | 26.7 | 0 | 19 | 29 | 27 | 27 | 27 | 463 | 29 |
| c49a | 19.5 | 0 | 30 | 32 | 30 | 35 | 36 | 578 | 36 |

Interpretation of the Results:

All the Examples according to the invention are clearly superior to the reference formulation as the reference forms do not achieve any measurable releases, or only slight ones.

As the content of active substance increases the in vitro release declines, as the amount of base is smaller for the same dose of active substance.

All the bases and other excipients are generally suitable, but display somewhat different release characteristics at comparable active substance concentrations. Examples c3, c4, c16, c21, c24, c25, c31, c32, c33, c46, c47, c49 and c49a have proved particularly suitable for the purposes of the invention.

The invention further relates to base-containing formulations for amphoteric active substances, such as for example telmisartan.

What is particularly surprising is the superiority of the forms according to the invention with amphoteric, i.e. acidically and basically soluble active substances, if one compares the in vitro releases of the pellets according to the invention on base starters with pellets in which the active substance has been applied to acid-containing cores. With a similar charge of amphoteric active substance is present the release of the base-containing pellets is significantly better than that of the acid-containing pellets, as shown by the comparison of the in vitro releases of Examples c10, c13, c14 and c15 (for releases see Table 2 and FIG. 1).

TABLE 2
Comparison of the in vitro releases and the characteristics
of area under the release curve (AUC) and maximum release
of pellets on base starters (Examples c3-c5) and acid starters
(Examples c13-c15 contain tartaric acid starter and Example
c10 contains succinic acid starter)

| | content of | in vitro | | | | | | | |
|---|---|---|---|---|---|---|---|---|---|
| | active | release after . . . minutes | | | | | | | maximum |
| Example | substance | 0 | 4 | 8 | 12 | 16 | 20 | AUC | release |
| c3 | 8.9 | 0 | 44 | 50 | 62 | 72 | 69 | 1046 | 72 |
| c4 | 23.2 | 0 | 18 | 14 | 17 | 17 | 14 | 293 | 18 |
| c5 | 29.4 | 0 | 9 | 9 | 8 | 8 | 6 | 147 | 9 |
| c13 | 3.4 | 0 | 16 | 17 | 17 | 17 | 18 | 302 | 18 |
| c14 | 9.5 | 0 | 7 | 8 | 8 | 7 | 8 | 134 | 8 |
| c15 | 17.4 | 0 | 4 | 4 | 3 | 3 | 3 | 63 | 4 |
| c10 | 19.7 | 0 | 0 | 0 | 0 | 0 | 0 | 0 | 0 |

The data show that Example c3 with approx 9% active substance content achieves a maximum release of 72%, whereas Example c13, also with approx 9% active substance content achieves a maximum release of only 8%, i.e. the maximum release with base starter is higher by about a factor of 9, and with regard to the AUC by a factor of 8. Example c4 with an active substance content of approx 23% achieves 18% release whereas Example c15 with an active substance content of only 17.4% achieves only 4% release, i.e. the maximum release and also the AUC value is higher by a factor of 5. Example c5 with an active substance content of 29% achieves 9% release and is thus comparable with Example c14 with an active substance content of only 9.5%. This means that e.g. for 1 capsule containing 80 mg of active substance for Example c5 only 235 mg of pellets are needed, which can very easily be packed into a size 3 capsule (approx 6*16 mm), whereas for 1 capsule containing 80 mg of active substance for Example c14, 762 mg of pellets are required, which can only be fitted into 2 size 1 capsules (approx 8*24 mm), i.e. the medication is substantially more difficult for the patient to take. Examples c15 with an active substance content of 17% achieve only 4 % maximum release and Example c10 (succinic acid starter with active substance content of 20%) does not release the active substance at all.

### BRIEF DESCRIPTION OF DRAWINGS

FIG. 1 shows the in vitro releases of the Examples c3-c5 according to the invention compared with the formulations on tartaric acid starters (Examples c13-c15) and on succinic acid starter (c10) with different contents of active substance in 0.005 mol citrate buffer pH 5.0.

FIG. 2 shows the schematic structure of a pharmaceutical composition according to the invention in the form of a sectional view of a pellet.

FIG. 3 shows the in vitro releases of the Examples c3-c5 according to the invention compared with the reference formulations c1-c2 with different contents of active substance in 0.005 mol citrate buffer pH 5.0.

FIG. 4 shows the in vitro releases of the Examples c33-c35 according to the invention compared with the reference formulation c28 with different contents of active substance in 0.01 mol phosphate buffer pH 5.0.

FIG. 5 shows the in vitro releases of the Examples c31 and c32 according to the invention compared with the reference formulation c28 with different contents of active substance in 0.01 mol phosphate buffer pH 5.0.

FIG. 6 shows the in vitro releases of the Examples c46 and c47 according to the invention compared with the reference formulations c43 and c45 with different contents of active substance in 0.0025 mol phosphate buffer pH 6.0.

FIG. 7 shows the in vitro releases of the Examples c49 and c49a according to the invention compared with the reference formulations c43 and c45 with different contents of active substance in 0.0025 mol phosphate buffer pH 6.0.

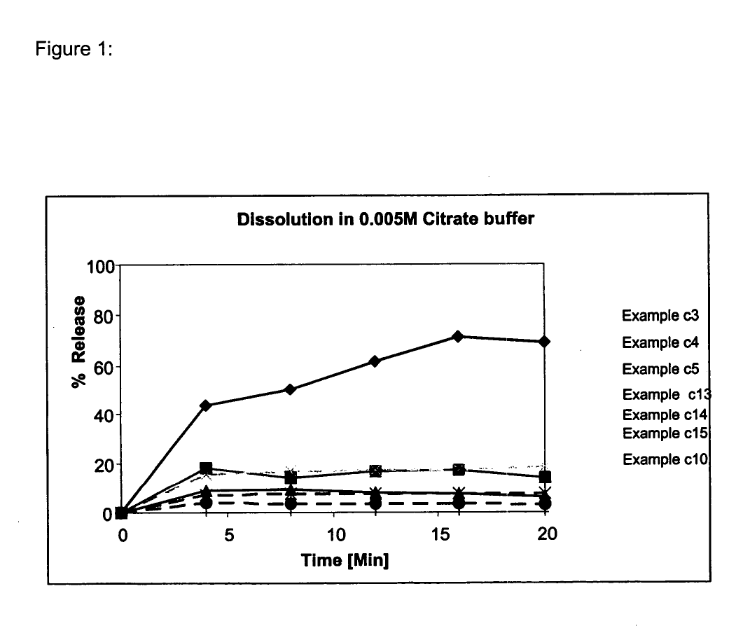

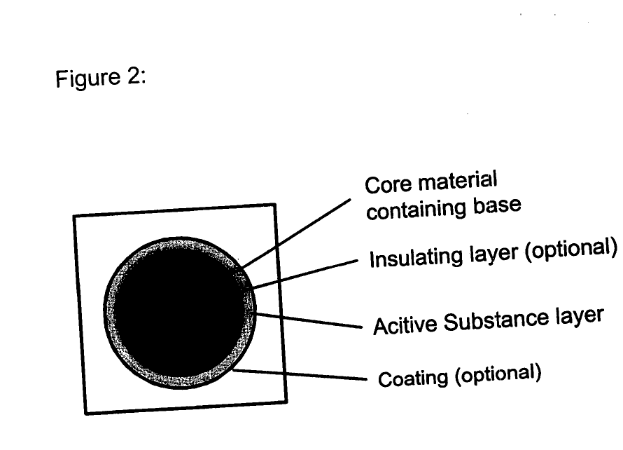

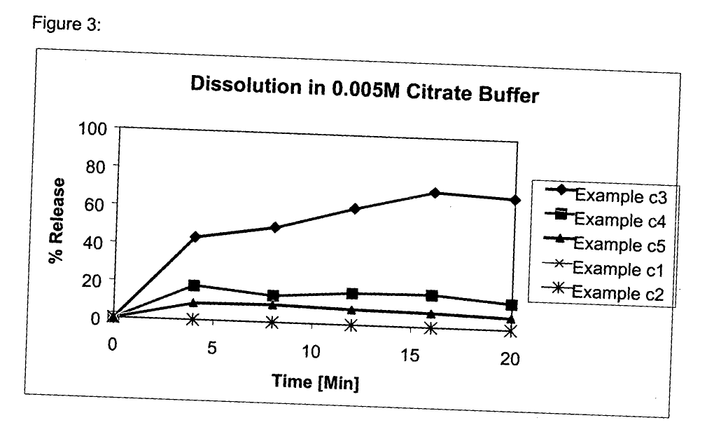

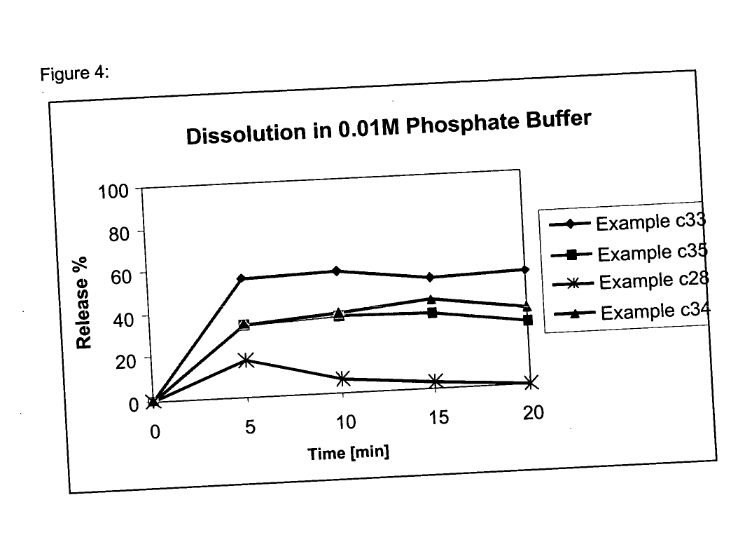

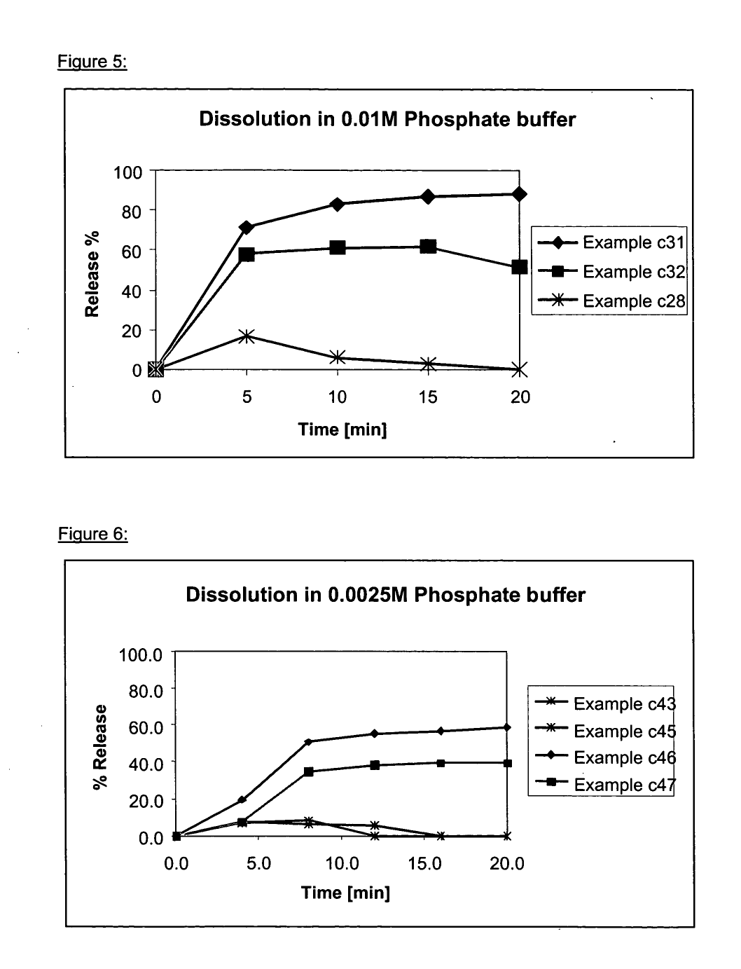

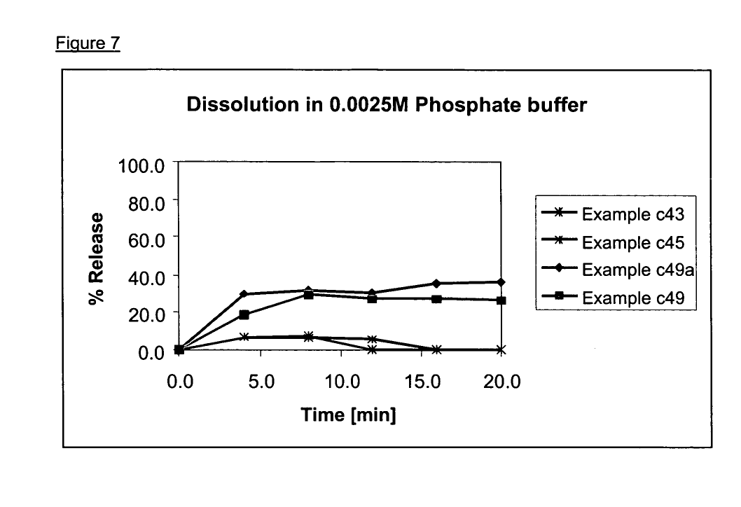

The Examples that follow are intended to illustrate the invention:

### EXAMPLES

The preparation of the following Examples usually takes place over 5 steps:
a: preparation of base-containing core material
b: isolation of the base-containing core material
c: preparation of the active substance layer
d: isolation of the pellets consisting of base-containing core
e: packing into capsules

Step b is absolutely essential if there is any incompatibility between base and active substance layer, otherwise this step may be omitted to simplify the production method. Step d is necessary if the mechanical stability of the active substance layer is insufficient to dissolve the active substance completely.

The brand names used in the Examples and not separately characterised refer to the following substances:

| | |
|---|---|
| Kollidon K25 | povidone (polyvinylpyrrolidone) |
| Avicel PH101 | microcrystalline cellulose |
| Klucel | hydroxypropylcellulose |

i.e. the Examples should be read as meaning that povidone, e.g. Kollidon K25 is used.
a: Examples of the Preparation of Base-containing Core Material

### Example a1

### Preparation of Meglumine-containing Core Material with Binder

Composition:

| | |
|---|---|
| Kollidon K25 | 3 parts by weight |
| Avicel PH101 | 20 parts by weight |
| meglumine | 77 parts by weight |

77 parts by weight of meglumine, 20 parts by weight of Avicel PH 101 and 3 parts by weight of Kollidon K25 are mixed for 15 minutes in a gyrowheel mixer. Then the powder mixture is transferred into a twin-screw metering device. This mixture is introduced into a twin-screw extruder of the Werner & Pfleiderer 37/18D type (or any other suitable type of extruder) at a speed of about 1 kg/h, together with water which is added by means of a ProMinent metering pump. The water is automatically regulated so as to obtain a nominal torque of approx. 19% in the extruder. The extrusion is carried out using a die plate with bores 8 mm in diameter.

The extruded strips are rounded off to form pellets in a WyPro Sphäromat, the process taking approx. 3 minutes at approx. 850 RPM.

Drying of the pellets at 80° C. for approx. 1.5 h in the GPCG1 fluidised bed dryer.

The core material is fractionated using a tumbler screening machine with different perforated plates having nominal mesh sizes of 0.71 to 1.25 mm. The suitable fractions of materials of between 0.71 and 0.90 or 0.90 and 1.12 mm are used in subsequent processes.

### Example a2

### Preparation of Meglumine-containing Core Material Without Binder

Composition:

| | |
|---|---|
| Avicel PH101 | 40 parts by weight |
| meglumine | 60 parts by weight |

60 parts by weight of meglumine and 40 parts by weight of Avicel PH 101 are mixed for 15 minutes in a gyrowheel mixer. Then the powder mixture is transferred into a twin-screw metering device. This mixture is introduced into a twin-screw extruder of the Werner & Pfleiderer 37/18D type (or any other suitable type of extruder) at a speed of about 1 kg/h, together with water which is added by means of a ProMinent metering pump. The water is automatically regulated so as to obtain a nominal torque of approx. 19% in the extruder. The extrusion is carried out using a die plate with bores 8 mm in diameter.

The extruded strips are rounded off to form pellets in a WyPro Sphäromat, the process taking about 3 minutes at approx. 850 RPM.

Drying of the pellets at 80° C. for approx. 1.5 h in the GPCG1 fluidised bed dryer.

The core material is fractionated using a tumbler screening machine with different perforated plates having nominal mesh sizes of 0.71 to 1.25 mm. The suitable fractions of materials of between 0.71 and 0.90 or 0.90 and 1.12 mm are used in subsequent processes.

### Example a3

### Preparation of Arginine-containing Core Material

Composition:

| | |
|---|---|
| Avicel PH101 | 40 parts by weight |
| arginine | 60 parts by weight |

Prepared analogously to Example a2

### Example a4

### Preparation of Trometamol-containing Core Material

Composition:

| | |
|---|---|
| Avicel PH101 | 40 parts by weight |
| trometamol | 60 parts by weight |

Prepared analogously to Example a2

### Example a5

### Preparation of Piperazine-containing Core Material

Composition:

| | |
|---|---|
| Avicel PH101 | 40 parts by weight |
| piperazine | 60 parts by weight |

Prepared analogously to Example a2

### Example a6

### Preparation of Sodium Hydroxide-containing Core Material

Composition:

| | |
|---|---|
| Avicel PH101 | 30 parts by weight |
| sodium hydroxide | 70 parts by weight |

Prepared by dissolving sodium hydroxide in water, then adding Avicel PH101. Further processing is carried out analogously to Example a2

### Example a7

### Preparation of Potassium Hydroxide-containing Core Material

Composition:

| | |
|---|---|
| Avicel PH101 | 40 parts by weight |
| potassium hydroxide | 60 parts by weight |

Prepared analogously to Example a6

### Example a8

### Preparation of Calcium Hydroxide-containing Core Material

Composition:

| | |
|---|---|
| Avicel PH101 | 70 parts by weight |
| Calcium hydroxide | 30 parts by weight |

Prepared analogously to Example a6

### Example a9

### Preparation of Sodium Hydroxide- and Meglumine-containing Core Material

Composition:

| | |
|---|---|
| Avicel PH101 | 30 parts by weight |
| sodium hydroxide | 20 parts by weight |
| meglumine | 50 parts by weight |

Prepared by dissolving sodium hydroxide in water, then adding Avicel PH101 and meglumine. Further processing is carried out analogously to Example a2

b: Example for the Isolation of the Base-containing Core Material

Composition:

| | |
|---|---|
| Base-containing core material | 23 parts by weight |
| Gum arabic | 1 part by weight |
| Talc | 2 parts by weight |

1 part by weight of gum arabic is dissolved with stirring in a mixture of 6.7 parts by weight of 96% ethanol and 13.5 parts by weight of purified water. Then 2 parts by weight of talc are dispersed in the solution with stirring.

In a fluidised bed processing plant, 23 parts by weight of base-containing core material are sprayed with the gum arabic/talc dispersion at an air entry temperature of 35°-40° C. by the under-bed spraying method.

The isolated base-containing core material is then dried at 40° C. in the circulating air dryer for 8 hours.

To remove lumps the dried isolated base-containing core material is screened through a screen with a nominal mesh size of 1.0 mm. The fraction of material (particle size less than 1 mm) is further processed.

c: Examples of the Preparation of the Active Substance Layer

The active substance layer is generally prepared in the same way, but with variations in the nature and quantity of the active substance, the nature and quantity of the binder, the amount of talc and isopropanol or the amount of ethanol. The preparation is therefore only described for Example c9, and the particular compositions for each active substance are shown in table form.

Preparation of Example c9:

Composition:

| | |
|---|---|
| isolated meglumine-containing core material | 12 parts by weight |
| hydroxypropylcellulose | 2.5 parts by weight |
| talc | 5 parts by weight |
| active substance (e.g. telmisartan) | 10 parts by weight |

Hydroxypropylcellulose is dissolved In 255 parts by weight of 2-propanol with stirring and then the active substance and talc are dispersed in this solution with stirring.

In a fluidised bed processing plant, 12 parts by weight of meglumine-containing core material are sprayed with the dispersion containing the active substance at an air entry temperature of 20°-30° C. by the under-bed spraying method.

The pellets containing the active substance are then dried at 35° C. in the circulating air dryer for 8 hours.

To remove lumps the pellets containing the active substance are screened through a screen with a nominal mesh size of 1.25 mm. The product fraction (particle size less than 1.25 mm) is further processed.

For other Examples of step c see below.

d: Example of the Isolation of the Pellets Containing Active Substance

Composition:

| | |
|---|---|
| pellets containing active substance | 23 parts by weight |
| gum arabic | 1 part by weight |
| talc | 2 parts by weight |

1 part by weight of gum arabic is dissolved with stirring in a mixture of 6.7 parts by weight of 96% ethanol and 13.5 parts by weight of purified water. Then 2 parts by weight of talc are dispersed in the solution with stirring.

In a fluidised bed processing plant, 23 parts by weight of pellets containing active substance are sprayed with the gum arabic/talc dispersion at an air entry temperature of 35°-40° C. by the under-bed spraying method.

The isolated meglumine-containing core material is then dried at 40° C. in the circulating air dryer for 8 hours.

To remove lumps the dried pellets containing active substance are screened through a screen with a nominal mesh size of 1.25 mm. The product fraction (particle size less than 1.25 mm) is further processed.

e) Packing into Capsules

A quantity of pellets containing active substance corresponding to the desired dosage in each case is packed into hard capsules, e.g. hard gelatine capsules, of suitable size using a capsule filling machine.

Other Examples of Compositions for Step c

The numbers given below, unless otherwise stated, are parts by weight. In each case parts by weight are specified which correspond to the active substance content determined experimentally, i.e. the spray losses, which may fluctuate somewhat from one batch to the next, were compensated in the calculation in each case so as to obtain truly comparable data.

For example, the 10-fold values may be regarded as amounts given in grams, i.e. for Example c1, 200.0 g of neutral pellets, 5.4 g of telmisartan, 1.4 g of povidone K90, 2.7 g of talc and 142.1 g of isopropanol.

The Examples which contain a commercially available neutral core instead of the base-containing starter cores according to the invention serve in each case as reference values for the in vitro testing.

### Telmisartan Examples

Examples c1-c2 contain a commercially available neutral core instead of the base-containing starter cores according to the invention. These cores serve as reference values for the in vitro testing (see Table 1). Examples c10-c15 comprise acid-containing cores instead of the base-containing starter cores according to the invention, so as to demonstrate the unforeseeable advantage of the base starters according to the invention over acid starters, which should theoretically be at least equally soluble, as telmisartan is also readily soluble in an acidic medium.

Example

| | c1 | c2 |
|---|---|---|
| active substance content (wt. %): | 2.60 | 16.80 |
| neutral pellets | 20.00 | 20.00 |
| telmisartan | 0.54 | 4.76 |
| povidone K 90 | 0.14 | 1.19 |
| talc | 0.27 | 2.38 |
| isopropanol (for preparation only) | 14.21 | 124.36 |

| | Example | | | | | | |
|---|---|---|---|---|---|---|---|
| | c3 | c4 | c5 | c6 | c7 | c8 | c9 |
| active | 8.9 | 23.2 | 29.4 | 33.7 | 10.6 | 14.5 | 33.7 |
| substance | | | | | | | |
| content | | | | | | | |
| (wt. %): | | | | | | | |
| meglumine | 12.00 | 12.00 | 12.00 | 12.00 | 12.00 | 12.00 | 12.00 |
| starter | | | | | | | |
| telmisartan | 1.26 | 4.68 | 7.28 | 9.88 | 1.56 | 2.34 | 9.88 |
| povidone | 0.32 | 1.17 | 1.82 | 2.47 | — | — | — |
| K 90 | | | | | | | |
| talc | 0.63 | 2.34 | 3.64 | 4.94 | 0.78 | 1.17 | 4.94 |
| Klucel | — | — | — | — | 0.39 | 0.59 | 2.47 |
| isopropanol | 32.94 | 122.27 | 190.19 | 258.12 | 40.37 | 60.55 | 255.65 |
| (for | | | | | | | |
| preparation | | | | | | | |
| only) | | | | | | | |

| | Example | | |
|---|---|---|---|
| | c10 | c11 | c12 |
| active substance content (wt. %): | 19.70 | 20.70 | 32.10 |
| succinic acid starter | 12.00 | 12.00 | 12.00 |
| telmisartan | 3.61 | 3.90 | 8.81 |
| talc | 1.81 | 1.95 | 4.41 |
| Klucel | 0.90 | 0.98 | 2.20 |
| isopropanol (for preparation only) | 93.51 | 100.91 | 228.06 |
| | Example | | |
| | c13 | c14 | c15 |
| active substance content (wt. %): | 3.40 | 9.50 | 17.40 |
| tartaric acid starter | 20.00 | 20.00 | 20.00 |
| telmisartan | 0.73 | 2.29 | 4.99 |
| talc | 0.36 | 1.14 | 2.50 |
| Klucel | 0.18 | 0.57 | 1.25 |
| isopropanol (for preparation only) | 18.84 | 59.20 | 129.17 |
| | Example | | |
| | c16 | c17 | c18 |
| active substance content (wt. %): | 19.70 | 28.50 | 32.90 |
| arginine starter | 12.00 | 12.00 | 12.00 |
| telmisartan | 3.60 | 6.80 | 9.30 |
| povidone K90 | 0.90 | 1.70 | 3.40 |
| talc | 1.80 | 3.40 | 4.65 |
| isopropanol (for preparation only) | 93.60 | 176.80 | 241.80 |

Example

| | c19 | c20 |
|---|---|---|
| active substance content (wt. %): | 17.10 | 26.20 |
| trometamol starter | 12.00 | 12.00 |
| telmisartan | 2.93 | 5.80 |
| povidone K 90 | 0.73 | 1.45 |
| talc | 1.47 | 2.90 |
| isopropanol (for preparation only) | 76.18 | 150.80 |

| | Example | | |
|---|---|---|---|
| | c21 | c22 | c23 |
| active substance content (wt. %): | 19.80 | 28.50 | 32.90 |
| lysine starter | 12.00 | 12.00 | 12.00 |
| telmisartan | 3.64 | 6.82 | 9.29 |
| povidone K90 | 0.91 | 1.71 | 3.32 |
| talc | 1.82 | 3.41 | 4.65 |
| isopropanol (for preparation only) | 94.64 | 177.32 | 241.54 |

| | Example | |
|---|---|---|
| | c24 | c25 |
| active substance content (wt. %): | 18.20 | 25.80 |
| sodium hydroxide starter | 12.00 | 12.00 |
| telmisartan | 3.20 | 5.63 |
| povidone K 90 | 0.80 | 1.41 |
| talc | 1.60 | 2.82 |
| isopropanol (for preparation only) | 83.20 | 146.38 |

| | Example | |
|---|---|---|
| | c26 | c27 |
| active substance content (wt. %): | 26.90 | 33.90 |
| potassium hydroxide starter | 12.00 | 12.00 |
| telmisartan | 6.12 | 9.98 |
| povidone K 90 | 1.53 | 2.50 |
| talc | 3.06 | 4.99 |
| isopropanol (for preparation only) | 159.12 | 259.48 |

### Meloxicam Examples:

| | Example | | |
|---|---|---|---|
| | c28 | c29 | c30 |
| active substance content (wt. %): | 14.80 | 25.20 | 33.00 |
| neutral pellets | 12.00 | 12.00 | 12.00 |
| meloxicam | 2.40 | 5.42 | 9.40 |
| povidone K90 | 0.60 | 1.36 | 2.35 |
| talc | 1.20 | 2.71 | 4.70 |
| isopropanol (for preparation only) | 62.40 | 140.92 | 244.40 |

Example

| | c31 | c32 |
|---|---|---|
| active substance content (wt. %): | 16.80 | 26.10 |
| meglumine starter | 12.00 | 12.00 |
| meloxicam | 2.85 | 5.78 |
| povidone K 90 | 0.71 | 1.45 |
| talc | 1.43 | 2.89 |
| isopropanol (for preparation only) | 74.10 | 150.28 |

| | Example | | |
|---|---|---|---|
| | c33 | c34 | c35 |
| active substance content (wt. %): | 17.10 | 28.60 | 33.30 |
| arginine starter | 12.00 | 12.00 | 12.00 |
| meloxicam | 2.93 | 6.87 | 9.58 |
| povidone K90 | 0.73 | 1.72 | 2.40 |
| talc | 1.47 | 3.44 | 4.79 |
| isopropanol (for preparation only) | 76.18 | 178.62 | 249.08 |

Example

| | c36 | c37 |
|---|---|---|
| active substance content (wt. %): | 15.90 | 25.70 |
| trometamol starter | 12.00 | 12.00 |
| meloxicam | 2.64 | 5.61 |
| povidone K 90 | 0.66 | 1.40 |
| talc | 1.32 | 2.81 |
| isopropanol (for preparation only) | 68.64 | 145.86 |

| | Example | | |
|---|---|---|---|
| | c38 | c39 | c40 |
| active substance content (wt. %): | 16.10 | 26.50 | 36.60 |
| lysine starter | 12.00 | 12.00 | 12.00 |
| meloxicam | 2.70 | 5.94 | 11.30 |
| povidone K90 | 0.68 | 1.49 | 2.83 |
| talc | 1.35 | 2.97 | 5.65 |
| isopropanol (for preparation only) | 70.20 | 154.44 | 293.80 |

Example

| | c41 | c42 |
|---|---|---|
| active substance content (wt. %): | 28.30 | 36.60 |
| calcium hydroxide starter | 12.00 | 12.00 |
| meloxicam | 6.75 | 12.21 |
| povidone K 90 | 1.69 | 3.05 |
| talc | 3.38 | 6.11 |
| isopropanol (for preparation only) | 175.50 | 317.46 |

### DT-TX 30-Examples

| | Example | | |
|---|---|---|---|
| | c43 | c44 | c45 |
| active substance content (wt. %): | 15.80 | 22.20 | 31.20 |
| neutral pellets | 12.00 | 12.00 | 12.00 |
| DT-TX 30 | 2.63 | 4.35 | 8.26 |
| povidone K90 | 0.66 | 1.09 | 2.07 |
| talc | 1.32 | 2.18 | 4.13 |
| isopropanol (for preparation only) | 68.38 | 113.10 | 214.76 |
| | Example | | |
| | c46 | c47 | c48 |
| active substance content (wt. %): | 16.30 | 25.70 | 33.60 |
| meglumine starter | 12.00 | 12.00 | 12.00 |
| DT-TX 30 | 2.73 | 5.60 | 9.76 |
| povidone K90 | 0.68 | 1.40 | 2.44 |
| talc | 1.37 | 2.80 | 4.88 |
| isopropanol (for preparation only) | 70.98 | 145.60 | 253.76 |
| | Example | | |
| | c49 | c49a | c50 |
| active substance content (wt. %): | 26.70 | 19.50 | 34.20 |
| arginine starter | 12.00 | 12.00 | 12.00 |
| DT-TX 30 | 6.00 | 4.00 | 10.20 |
| povidone K 90 | 1.50 | 1.50 | 2.55 |
| talc | 3.00 | 3.00 | 5.10 |
| isopropanol (for preparation only) | 156.00 | 156.00 | 265.20 |
| | Example | | |
| | c51 | c52 | c53 |
| active substance content (wt. %): | 15.70 | 26.00 | 37.60 |
| lysine starter | 12.00 | 12.00 | 12.00 |
| DT-TX 30 | 2.59 | 5.74 | 13.20 |
| povidone K90 | 0.65 | 1.44 | 3.30 |
| talc | 1.30 | 2.87 | 6.60 |
| isopropanol (for preparation only) | 67.34 | 149.24 | 343.20 |

### Gliquidone Examples

| | Example | | |
|---|---|---|---|
| | c54 | c55 | c56 |
| active substance content (wt. %): | 14.80 | 21.70 | 31.90 |
| neutral pellets | 12.00 | 12.00 | 12.00 |
| gliquidone | 2.40 | 4.21 | 8.67 |
| povidone K90 | 0.60 | 1.05 | 2.17 |
| talc | 1.20 | 2.11 | 4.34 |
| isopropanol (for preparation only) | 62.40 | 109.46 | 225.42 |
| | Example | | |
| | c57 | c58 | c59 |
| active substance content (wt. %): | 15.60 | 22.30 | 31.20 |
| meglumine starter | 12.00 | 12.00 | 12.00 |
| gliquidone | 2.57 | 4.38 | 8.26 |
| povidone K90 | 0.64 | 1.10 | 2.07 |
| talc | 1.29 | 2.19 | 4.13 |
| isopropanol (for preparation only) | 66.82 | 113.88 | 214.76 |

Example

| | c60 | c61 |
|---|---|---|
| active substance content (wt. %): | 17.80 | 27.10 |
| arginine starter | 12.00 | 12.00 |
| gliquidone | 3.10 | 6.20 |
| povidone K 90 | 0.78 | 1.55 |
| talc | 1.55 | 3.10 |
| isopropanol (for preparation only) | 80.60 | 161.20 |

| | Example | | |
|---|---|---|---|
| | c62 | c63 | c64 |
| active substance content (wt. %): | 14.90 | 25.00 | 32.60 |
| lysine starter | 12.00 | 12.00 | 12.00 |
| gliquidone | 2.41 | 5.32 | 9.12 |
| povidone K90 | 0.60 | 1.33 | 2.28 |
| talc | 1.21 | 2.66 | 4.56 |
| isopropanol (for preparation only) | 62.66 | 138.32 | 237.12 |

### Repaglinide Examples

| | Example | | |
|---|---|---|---|
| | c65 | c66 | c67 |
| active substance content (wt. %): | 4.30 | 11.00 | 16.50 |
| neutral pellets | 12.00 | 12.00 | 12.00 |
| repaglinide | 0.56 | 1.63 | 2.78 |
| povidone K90 | 0.14 | 0.41 | 0.70 |
| talc | 0.28 | 0.82 | 1.39 |
| isopropanol (for preparation only) | 14.56 | 42.38 | 72.28 |
| | Example | | |
| | c68 | c69 | c70 |
| active substance content (wt. %): | 3.40 | 11.40 | 13.50 |
| meglumine starter | 12.00 | 12.00 | 12.00 |
| repaglinide | 0.44 | 1.70 | 2.12 |
| povidone K90 | 0.11 | 0.43 | 0.53 |
| talc | 0.22 | 0.85 | 1.06 |
| isopropanol (for preparation only) | 11.44 | 44.20 | 55.12 |

Example

| | c71 | c72 |
|---|---|---|
| active substance content (wt. %): | 5.10 | 8.00 |
| arginine starter | 12.00 | 12.00 |
| repaglinide | 0.67 | 1.11 |
| povidone K 90 | 0.17 | 0.28 |
| talc | 0.34 | 0.56 |
| isopropanol (for preparation only) | 17.42 | 28.86 |

| | Example | | |
|---|---|---|---|
| | c73 | c74 | c75 |
| active substance content (wt. %): | 5.00 | 8.60 | 15.00 |
| lysine starter | 12.00 | 12.00 | 12.00 |
| repaglinide | 0.66 | 1.21 | 2.44 |
| povidone K90 | 0.17 | 0.30 | 0.61 |
| talc | 0.33 | 0.61 | 1.22 |
| isopropanol (for preparation only) | 17.16 | 31.46 | 63.44 |

1. A pharmaceutical composition for oral administration with a bioavailability which is substantially independent of the gastric pH, comprising a plurality of pellets synthesised in each case from:
a) a core material;
b) an optional insulating layer;
c) an active substance layer which has pH-dependent solubility characteristics and a dose number of significantly greater than 1 at a pH of less than 7 or one of the physiologically acceptable salts thereof; and
d) an optional coating,
wherein the core material consists of one or more pharmaceutically acceptable inorganic or organic base(s) with a water solubility of more than 1 g/250 ml at 20° C., optionally with the addition of binders or other adjuvants.

2. A pharmaceutical composition for oral administration with a bioavailability which is substantially independent of the gastric pH, comprising a plurality of pellets synthesised in each case from:
a) a core material;
b) an optional insulating layer;
c) an active substance layer which has pH-dependent solubility characteristics and a dose number of significantly greater than 1 at a pH of less than 6 or one of the physiologically acceptable salts thereof; and
d) an optional coating,
wherein the core material consists of one or more pharmaceutically acceptable inorganic or organic base(s) with a water solubility of more than 1 g/250 ml at 20° C., optionally with the addition of binders or other adjuvants.

3. The pharmaceutical composition according to claim 1, wherein the pharmaceutically acceptable base is NaOH, KOH, Ca(OH)₂, Na₂CO₃, ammonia, diethanolamine, meglumine, lysine, arginine, ethanolamine, piperazine, triethanolamine or trometamol.

4. The pharmaceutical composition according to claim 2, wherein the pharmaceutically acceptable base is NaOH, KOH, Ca(OH)₂, Na₂CO₃, ammonia, diethanolamine, meglumine, lysine, arginine, ethanolamine, piperazine, triethanolamine or trometamol.

5. The pharmaceutical composition according to claim 3, characterised in that the pharmaceutically acceptable organic base is meglumine, lysine, arginine, trometamol.

6. The pharmaceutical composition according to claim 4, characterised in that the pharmaceutically acceptable organic base is meglumine, lysine, arginine, trometamol.

7. The pharmaceutical composition according to claim 5, characterised in that the pharmaceutically acceptable organic base is meglumine.

8. The pharmaceutical composition according to claim 6 characterised in that the pharmaceutically acceptable organic base is meglumine.

9. The pharmaceutical composition according to claim 1, wherein the active substance is telmisartan, meloxicam, DT-TX 30 SE, BIBV 308 SE (terbogrel), AGEE 623 (repaglinide), gliquidone or glibenclamide or a physiologically acceptable salt thereof.

10. The pharmaceutical composition according to claim 2, wherein the active substance is telmisartan, meloxicam, DT-TX 30 SE, BIBV 308 SE (terbogrel), AGEE 623 (repaglinide), gliquidone or glibenclamide or a physiologically acceptable salt thereof.

11. The pharmaceutical composition according to claim 1, wherein the binder is selected from the group comprising the hydroxypropylcelluloses, the hydroxypropylmethylcelluloses, the methylcelluloses, the hydroxyethylcelluloses, the carboxymethylcelluloses, the polyvinylpyrrolidones, the copolymers of N-vinylpyrrolidone and vinyl acetate or combinations of these polymers.

12. The pharmaceutical composition according to claim 2, wherein the binder is selected from the group comprising the hydroxypropylcelluloses, the hydroxypropylmethylcelluloses, the methylcelluloses, the hydroxyethylcelluloses, the carboxymethylcelluloses, the polyvinylpyrrolidones, the copolymers of N-vinylpyrrolidone and vinyl acetate or combinations of these polymers.

13. The pharmaceutical composition according to claim 1, wherein the core material has an average particle size of 0.4 to 1.5 mm.

14. The pharmaceutical composition according to claim 2, wherein the core material has an average particle size of 0.4 to 1.5 mm.

15. The pharmaceutical composition according to claim 1, wherein the insulating layer consists of a water-soluble polymer, optionally with the addition of suitable plasticisers, separating agents and pigments.

16. The pharmaceutical composition according to claim 2, wherein the insulating layer consists of a water-soluble polymer, optionally with the addition of suitable plasticisers, separating agents and pigments.

17. The pharmaceutical composition according to claim 15, wherein the water-soluble polymer consists of gum arabic or a partially or totally synthetic polymer selected from among the hydroxypropylcelluloses, the hydroxypropylmethylcelluloses, the methylcelluloses, the hydroxyethylcelluloses, the carboxymethylcelluloses, the polyvinylpyrrolidones, the copolymers of N-vinylpyrrolidone and vinyl acetate or combinations of these polymers.

18. The pharmaceutical composition according to claim 16, wherein the water-soluble polymer consists of gum arabic or a partially or totally synthetic polymer selected from among the hydroxypropylcelluloses, the hydroxypropylmethylcelluloses, the methylcelluloses, the hydroxyethylcelluloses, the carboxymethylcelluloses, the polyvinylpyrrolidones, the copolymers of N-vinylpyrrolidone and vinyl acetate or combinations of these polymers.

19. The pharmaceutical composition according to claim 1, wherein the coating consists of film-forming agents, plasticisers and optionally pigments.

20. The pharmaceutical composition according to claim 2, wherein the coating consists of film-forming agents, plasticisers and optionally pigments.

21. The pharmaceutical composition according to claim 1, wherein the pellets containing active substance are packed into hard capsules.

22. The pharmaceutical composition according to claim 2, wherein the pellets containing active substance are packed into hard capsules.

23. Process for preparing a pharmaceutical composition for oral administration containing an active substance with pH-dependent solubility characteristics and a dose number of significantly more than 1 at a pH of less than 7 or one of the physiologically acceptable salts thereof, comprising the steps of:
a) synthesising the core material from one or more pharmaceutically acceptable inorganic or organic base(s) with a water solubility of more than 1 g/250 ml at 20° C., optionally with the addition of binders or other adjuvants, in by pan methods, on pelleting plates or by extrusion/spheronisation,
b) applying an insulating layer consisting of one or more water-soluble, pharmaceutically acceptable polymers, optionally with the addition of plasticisers, separating agents and/or pigments, to the core material,
c) applying the active substance from a dispersion containing binder and optionally separating agent, and simultaneously or subsequently drying to eliminate the dispersing agent,
d) optionally applying a coating of film-forming agents, plasticisers and optionally pigments and
e) packing the pellets containing active substance thus obtained into hard capsules.
